(19) United States
(12) Patent Application Publication
(10) Pub. No.: US 2024/0058042 A1
(43) Pub. Date: Feb. 22, 2024

(54) SURGICAL OSTEOTOMY FIXATION DEVICES

(71) Applicant: The Research Foundation for the State University of New York, Albany, NY (US)

(72) Inventor: Timothy Damron, LaFayette, NY (US)

(21) Appl. No.: 18/236,678

(22) Filed: Aug. 22, 2023

Related U.S. Application Data
(60) Provisional application No. 63/399,751, filed on Aug. 22, 2022.

(51) Int. Cl. A61B 17/80

(52) U.S. Cl. CPC A61B 17/8061; A61B 17/8085; A61B 2017/564

(57) ABSTRACT

A surgical osteotomy fixation device having a first brace portion with a first plurality of openings formed therethrough, and a second brace portion with a second plurality of openings formed therethrough. A bridge portion extends between the first brace portion and the second brace portion, the bridge portion including at least one hole formed therethrough. The device is fastened in place to allow fixation of a tibial tubercle fragment during osteotomies during complex primary and revision total knee surgery, as well as sports and reconstructive procedures.

### CROSS-REFERENCE TO RELATED APPLICATION

This application claims the benefit of U.S. Provisional Application No. 63/399,751, filed Aug. 22, 2022, the entirety of which is hereby incorporated herein by this reference.

### BACKGROUND OF THE INVENTION

### 1. Field of the Invention

The disclosure relates generally to surgical devices. More particularly, the present invention relates to surgical osteotomy fixation devices.

### 2. Description of the Related Art

The patellar tendon inserts anteriorly on the tibial tubercle/tuberosity in the proximal tibia. It is situated at the lower end of the extensor mechanism, which consists of the quadriceps muscles in the thigh, the quadriceps tendon, patella, and patellar tendon with the described insertion on the tibia. Disruption of any portion of this mechanism can lead to compromise or complete loss of the ability to straighten the knee. However, in some orthopedic conditions, the tuberosity with its intact insertion of the patellar tendon must be lifted up and mobilized either for exposure of the inner aspect of the bone of the proximal tibia during complex primary or revision total knee arthroplasties/replacements (in order to avoid rupture of the soft tissue insertion of the patellar tendon from the bone, which is particularly difficult to repair, or to expose an intact tibial component of a total knee replacement that must be removed or revised during revision total knee surgery) or during procedures to address instability of the patella relative to the femur (subluxation or dislocation of the patella).

In all cases, the cut portion of the bone completed by the osteotomy should optimally be placed back into the native tibia either in an anatomically reduced position or in the position desired by the surgeon in order to facilitate healing and restoration of the normal anatomy of the proximal tibia and extensor mechanism. Secure fixation of the fragment is essential to allow healing of the fragment bone to the intact host bone. In some situations, particularly those of revision and complex primary total knee arthroplasty, screws through the fragment of the osteotomy into the posterior cortex of the tibia, which are the most common technique now used in non-arthroplasty indications, are not able to be placed since the intramedullary portion of the tibial component is in the way, blocking placement of the anterior to posteriorly directed screws into the intact posterior cortex. Other alternatives are sutures or wires through holes in the thin proximal metaphyseal bone and then passed over or through the tibial tubercle osteotomy fragment.

However, these sutures/wires are not only difficult to pass through the medial and lateral intact bone holes as well as the tuberosity fragment, the intact bone holes medial and lateral to the osteotomy needed to be created to pass the sutures or wires are difficult to create reliably without breaking through the bone, and the sutures/wires have a tendency to cut through the bone at the edge of those holes, all of which may compromise fixation of the fragment. Moreover, these techniques provide tenuous fixation at best.

Thus, standard techniques that include screw fixation alone or fixation with wire or suture, none of which are reliable in holding the bone in place to avoid displacement post-operatively and possibly leading to non-union, malunion, extensor weakness, extensor lag, or complete loss of active knee extension. Multiple other anatomy specific plate/screw fixation devices exist that are used in other sites (e.g. proximal and distal humerus and femur, cervical/thoracic/lumbar spine, foot, hand, calcaneus, etc.), but none for this specific indication. It is thus to address this problem with the prior art that the present invention is primarily directed.

### BRIEF SUMMARY OF THE INVENTION

Briefly described, the present invention improves fixation of the tibial tubercle fragment during osteotomies of the region for exposure during complex primary and revision total knee surgery, as well as sports and reconstructive procedures necessitating transfer of the tibial tubercle for patellofemoral instability problems.

In one embodiment, a first aspect of the disclosure provides a surgical fixation device. The device includes a first brace portion including a first end, a second end formed opposite the first end, and a first plurality of openings formed through the first brace portion, the first plurality of openings extending between the first end and the second end of the first brace portion, a second brace portion positioned opposite the first brace portion. The second brace portion includes a first end, a second end formed opposite the first end, and a second plurality of openings formed through the second brace portion, the second plurality of openings extending between the first end and the second end of the second brace portion. There is a bridge portion extending between the first brace portion and the second brace portion, the bridge portion including at least one hole formed therethrough.

The present invention is advantageous in that it allows the fixation of a osteotomy fragment during osteotomies for improved healing and more successful medical treatment. The invention has industrial applicability as it provides a medical device for use in treating patients with bone-related issues and injuries. Further illustrative aspects of the present disclosure are designed to solve the problems herein described and/or other problems as would be apparent to one of skill in the art.

### BRIEF DESCRIPTION OF THE DRAWINGS

FIG. 1 shows a front view of a surgical fixation device, according to embodiments of the disclosure.

FIG. 2 shows a side view of the surgical fixation device of FIG. 1, according to embodiments of the disclosure.

FIG. 3 shows a bottom view of the surgical fixation device of FIG. 1, according to embodiments of the disclosure.

FIG. 4 shows a front view of the surgical fixation device attached to a patient's tibia undergoing an osteotomy process, according to embodiments of the disclosure.

FIG. 5 shows a side view of the surgical fixation device and patient's tibia undergoing the osteotomy process shown in FIG. 4, according to embodiments of the disclosure.

FIG. 6 show front views of a surgical fixation device, according to additional embodiments of the disclosure.

FIG. 7 show front views of a surgical fixation device, according to additional embodiments of the disclosure.

FIG. 8 shows a front view of a surgical fixation device, according to another embodiment of the disclosure.

FIG. 9 shows a front exploded view of the surgical fixation device of FIG. 8, according to embodiments of the disclosure.

FIG. 10 shows a front view of a surgical fixation device, according to further embodiments of the disclosure.

### DETAILED DESCRIPTION

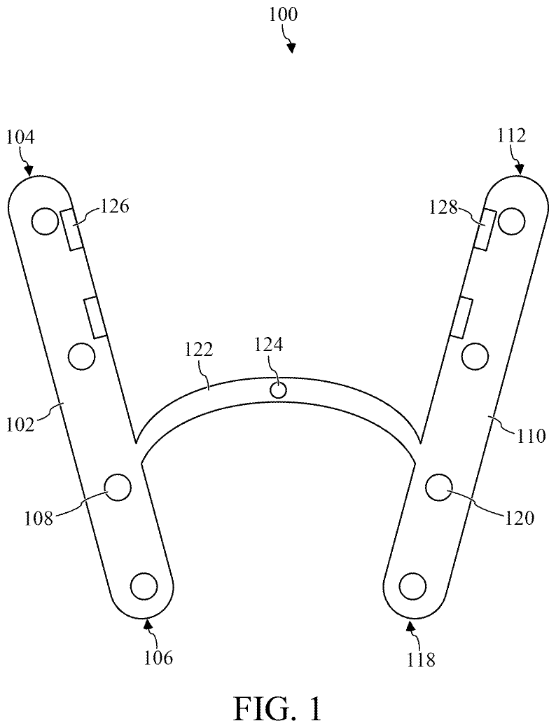

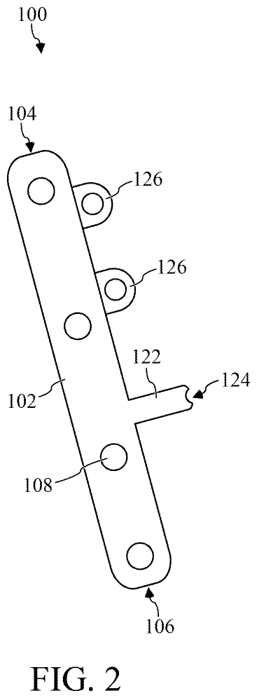

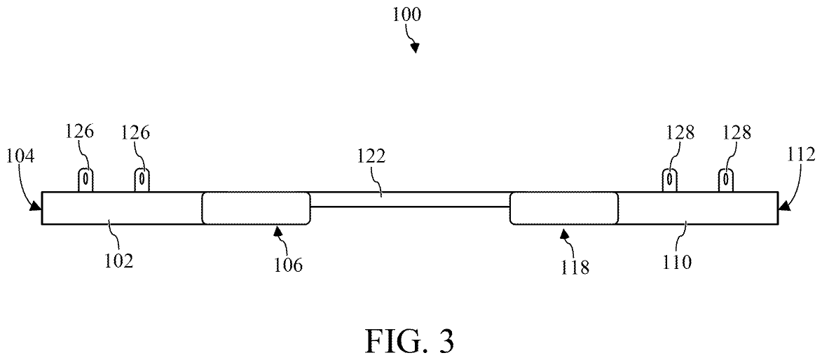

With reference to the figures in which like numerals represent like elements throughout the several views, FIGS. 1-3 show various views of a surgical fixation device 100. More specifically, FIG. 1 shows a front view of surgical fixation device 100, FIG. 2 shows a side view of surgical fixation device 100, and FIG. 3 shows a bottom view of surgical fixation device 100. In the non-limiting example, surgical fixation device 100 may include a first brace portion 102. First brace portion 102 may include a first end 104, and a second end 106 formed opposite first end 104. Additionally, first brace portion 102 may also include a first plurality of openings 108 formed therethrough. More specifically, a first plurality of openings 108 may be formed through a front surface of first brace portion 102.

As shown in FIG. 1, the first plurality of openings 108 may be formed through first brace portion 102 and may be spaced apart and/or extend between first end 104 and second end 106 of first brace portion 102. In the non-limiting example shown, first brace portion 102 may include four openings 108 formed therethrough. However, the number of openings 108 included in or formed through first brace portion 102 is illustrative. As such, it is understood that first brace portion 102 may include more or less openings 108. As discussed herein, each of the plurality of openings 108 may receive a screw to secure fixation device 100 to a patient's bone after performing the osteotomy processes discussed herein.

Also shown in FIG. 1, first brace portion 102 may be angled relative to other portions of surgical fixation device 100 (e.g., second brace portion 110). However, it is understood that the angle, tilt, and/or orientation of first brace portion 102, as shown in FIG. 1, is illustrative, and first brace portion 102 may be oriented in any suitable angle or position to aid in the osteotomy processes discussed herein.

Additionally as shown in FIGS. 1-3, surgical fixation device 100 may also include a second brace portion 110. Second brace portion 110 may be positioned opposite first brace portion 102. That is, second brace portion 110 may be positioned opposite to and separated/spaced-apart from first brace portion 102. Similar to first brace portion 102, second brace portion 110 may include a first end 112, and a second end 118 formed opposite first end 112. Additionally, second brace portion 110 may also include a second plurality of openings 120 formed therethrough. More specifically, a second plurality of openings 120 may be formed through a front surface of second brace portion 110. As shown in FIG. 1, the second plurality of openings 120 may be formed through second brace portion 110 and may be spaced apart and/or extend between first end 112 and second end 118 of second brace portion 110. In the non-limiting example shown, and similar to first brace portion 102, second brace portion 110 may include four openings 120 formed therethrough. However, the number of openings 120 included in or formed through second brace portion 110 is illustrative. As such, it is understood that second brace portion 110 may include more or less openings 120. As discussed herein, each of the plurality of openings 120 may receive a screw to secure fixation device 100 to a patient's bone after performing the osteotomy processes discussed herein.

Also shown in FIG. 1, second brace portion 110 may be angled relative first brace portion 102 of surgical fixation device 100 (e.g., second brace portion 110). More specifically, first brace portion 102 and second brace portion 110 may be angled, tilted, and/or oriented with respect one another such that a distance between first end 104 of first brace portion 102 and first end 112 of second brace portion 110 may be greater than a distance between second end 106 of first brace portion 102 and second end 118 of second brace portion 110. However, it is understood that first brace portion 102 and/or second brace portion 110 may be oriented in any suitable angle or position to aid in the osteotomy processes discussed herein.

Surgical fixation device 100 may also include a bridge portion 122. As shown in FIG. 1, bridge portion 122 may extend between first brace portion 102 and second brace portion 110. More specifically, bridge portion 122 may extend between, may extend from, and/or may connect first brace portion 102 and second brace portion 110 of surgical fixation device 100. Additionally, bridge portion 102 may be positioned substantially between first end 104 and second end 106 of first brace portion 102, and first end 112 and second end 118 of second brace portion 110, respectively. In the non-limiting example, bridge portion 122 may be formed substantially central to first brace portion 102/second brace portion 110, and/or may be centrally located between first end 104/first end 112 and second end 106/second end 118. However, it is understood that bridge portion 122 may be formed or positioned closer to or further from first end 104/first end 112 and second end 106/second end 118.

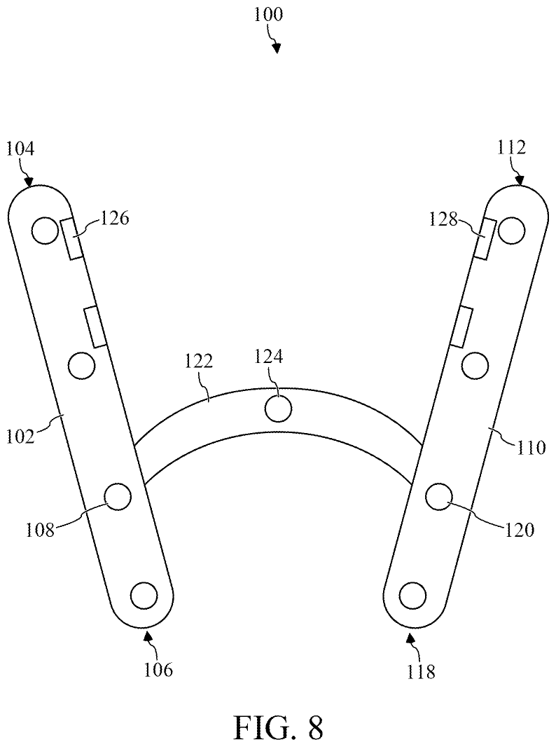

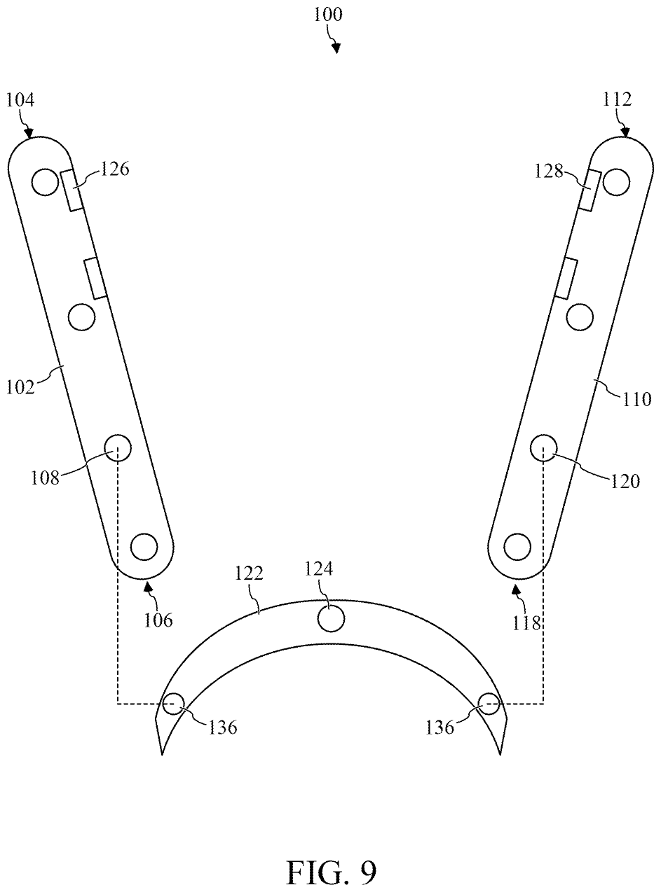

As shown in FIGS. 1-3, bridge portion 122 may be formed integral with first brace portion 102 and second brace portion 110. That is, first brace portion 102, second brace portion 110, and bridge portion 122 may be formed integral with one another and/or may be formed as single, unitary body or device. In this example, surgical fixation device 100 may be cut, punched, and/or formed from a single sheet of material, as discussed herein. In other non-limiting examples, first brace portion 102, second brace portion 110, and bridge portion 122 may be formed as distinct components that may be coupled to one another (see, FIGS. 8 and 9), as discussed herein.

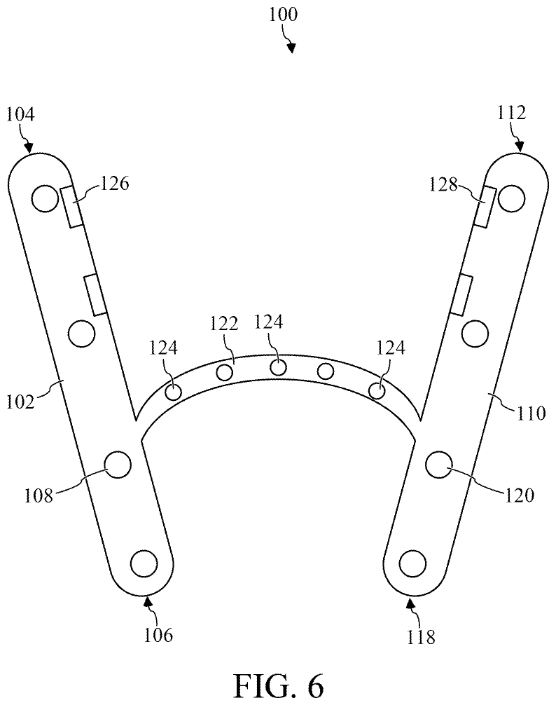

Bridge portion 122 of surgical fixation device 100 may also include at least one hole 124 formed therethrough, or include a similar bridge fastening point that allows physical fixation of the bridge portion 122. In the example, a single hole 124 may be positioned or formed through bride portion 122 centrally between first brace portion 102 and second brace portion 110. Although a single hole 124 is shown in bridge portion 122, it is understood that bridge portion 122 may include more holes 124 extending across the length of bridge portion 122 (see, FIG. 6), as discussed herein. Similar to openings 108, 120, hole(s) 124 may receive a screw to secure fixation device 100 to a patient's bone after performing the osteotomy processes discussed herein. Furthermore, opening 124 can be enlarged so that it is similar in size to openings 108 and 120, which would necessitate broadening bridge portion 122 only around the opening 124, maintaining the remainder of bridge portion 122 relatively thin. This provides the ability to contour this portion to the bone surface. Other fastening points or means can be used in the same manner as the hole(s) 124 to allow the fixed attachment of a wire or suture.

In the non-limiting example shown in FIGS. 1-3, surgical fixation device 100 may also include at least one first suture loop 126, and at least one second suture loop 128. More specifically, first brace portion 102 may include at least one first suture loop 126 formed adjacent first end 104, while second brace portion 110 may include at least one second suture 128 formed adjacent first end 112. Additionally as shown, first suture loop(s) 126 may be formed adjacent bridge portion 122 and/or may be formed on a side of first brace portion 102 closest to second brace portion 110. Similarly, second suture loop(s) 128 may be formed adjacent bridge portion 122 and/or may be formed on a side of second brace portion 110 closest to first brace portion 102. In the example shown in FIGS. 1-3, first suture loop(s) 126 and second suture loops(s) 128 may be formed integral with first brace portion 102 and second brace portion 110, respectively. In other non-limiting examples (not shown), first suture loop(s) 126 and second suture loops(s) 128 may be coupled to or affixed to first brace portion 102 and second brace portion 110, respectively.

With reference to FIGS. 1 and 3, first suture loop(s) 126 and second suture loops(s) 128 may extend away from (e.g., substantially perpendicular to) the front face of first brace portion 102 or second brace portion 110. However, and as discussed herein, the positioned, orientation, and/or angle of first suture loop(s) 126 and second suture loops(s) 128 may be adjusted as a result of the material forming each suture loop. In the non-limiting example two first suture loops 126 and two second suture loops 128 are shown. However, the number of suture loops 126, 128 shown are illustrative, and first brace portion 102/second brace portion 110 may include more less suture loops than shown. As discussed herein, suture loops 126, 128 may aid securing and selectively fastening a portion of the patient's bone using sutures after performing the osteotomy processes, as discussed herein.

As shown in FIGS. 1-3, surgical fixation device 100 may be formed as single, unitary device. More specifically, first brace portion 102, second brace portion 110, bridge portion 122, first suture loop(s) 126, and second suture loop(s) 128 may all be integrally formed as a single, unitary device and/or all portions may be formed from a single, continuous piece of material. In the non-limiting example, surgical fixation device 100, and all its portions, may be formed from a substantially malleable material, such that the distinct portions of surgical fixation device 100 may be flexed, bent, and/or manipulated into a desired shape. Once bent or manipulated, surgical fixation device 100, and its manipulated portions, may remain in the desired shape or position. In non-limiting examples, surgical fixation device 100 may be formed from metal, metal-alloy, polymers, or similar material having similar material characteristics and/or properties.

It should be appreciated that the number of suture loops 126 can vary, as well as the configuration of suture loops 126 which can also vary, such as using a flatter, more contoured configuration. Further variations in the spatial orientation of suture loop 126 and 128 with openings 108 and 120, respectively, and lengths of the plates 102 and 110, and the number of holes/openings 108 and 120 in each plate are possible.

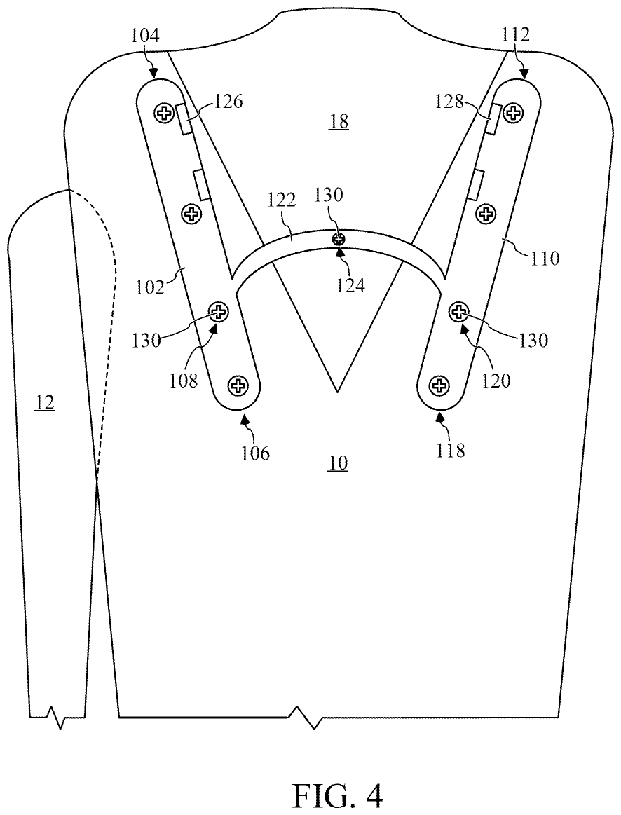

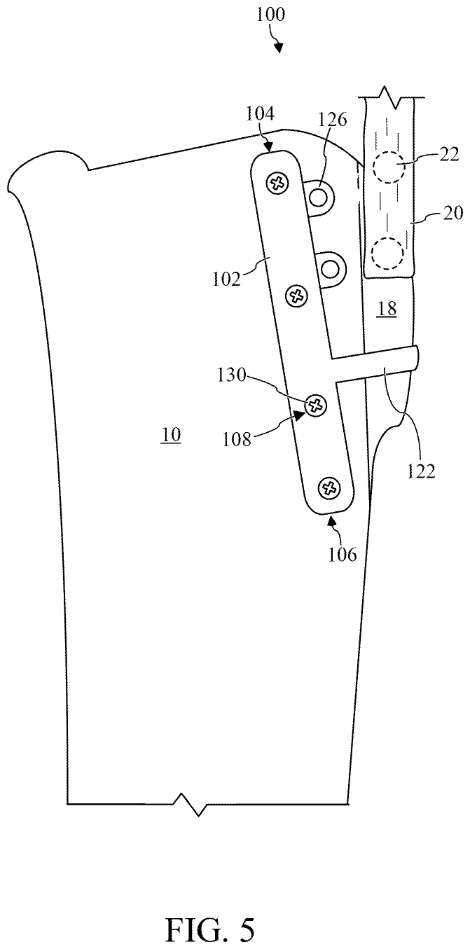

Turning to FIGS. 4 and 5, surgical fixation device 100 is shown as being coupled and/or a fixed to a patient's bone. More specifically, FIG. 4 shows a front view of a patient's tibia 10 and fibula 12, and FIG. 5 shows a side view of patient's tibia 10 in fibula 12. As discussed herein, surgical fixation device 100 may be coupled or selectively fastened to a portion of patient's tibia 10 after performing an osteotomy process on the patient. It is understood that similarly numbered and/or named components may function in a substantially similar fashion. Redundant explanation of these components has been omitted for clarity.

With reference to FIG. 4, surgical fixation device 100 may be positioned adjacent to and/or may at least partially cover a portion of the section 18 of tibia 10 removed during the osteotomy procedure. More specifically, first brace portion 102 may be positioned adjacent a first side of removed section 18, and second brace portion 110 may be positioned adjacent a second side of removed section 18, opposite the first side/first brace portion 102. Additionally as shown, bridge portion 122 may extend between first brace portion 102 and second brace portion 110, and may extend over at least a portion of removed section 18 of tibia 10. After performing the osteotomy procedure, it may be the physician's/surgeon's responsibility to reposition removed section 18 into the remaining portion of tibia 10/the portion in which section 18 was removed. Because of its malleable properties, as discussed herein, surgical fixation device 100 may bend, be shaped, and/or be manipulated to correspond to the shape of tibia 10 and removed section 18. That is, the shape or configuration of first brace portion 102, second brace portion 110, and/or bridge portion 122 may be manipulated search that each portion of surgical fixation device 100 corresponds to, contours, and/or compliments the shape of tibia 10 and removed section 18 as to not change or disrupt the natural anatomy of tibia 10.

To secure or reposition section 18 back in tibia 10 to allow for proper healing post osteotomy procedure, surgical fixation device 100 may be attached to tibia 10 and/or section 18. In the non-limiting example shown in FIGS. 4 and 5, surgical fixation device 100 may be coupled, secured, fastened and/or affixed to tibia 10 and/or section 18 using screws 130. screws 130 may be positioned through each of the plurality of openings 108, 120 and/or hole(s) 124 formed in/through surgical fixation device 100. For example, screw 130 may be positioned through each of the plurality of openings 108 formed through first brace portion 102 of surgical fixation device 100, and may be attached, embedded, and/or affixed to a portion of tibia 10 positioned adjacent section 18. Similarly, screw 130 may be positioned through each of the plurality of openings 120 formed through second brace portion 1/10 of surgical fixation device 100. Each screw positioned through openings 120 may the attached, embedded, and/or affixed 2A portion of tibia 10 positioned adjacent section 18, and opposite first brace portion 102. Furthermore, and as shown in FIG. 4, screw 130 may be positioned through hole 124 formed through bridge portion 122 of surgical fixation device 100. Screw 130 position through hole 124 may be attached, embedded, and/or affixed to section 18 of tibia 10 formed between first brace portion 102 and second brace portion 110. Although shown and discussed herein as a screw, it is understood that any suitable component may be used to secure, affix, and/or couple surgical fixation device 100 to tibia 10/removed section 18, as discussed herein.

Turning to FIG. 5, a tendon 20 of the patient is also shown. In the non-limiting example, tendon 20 may be positioned over and/or may substantially cover at least a portion of removed section 18 of tibia 10. To aid in the retention of removed section 18 in tibia 10 by affixing surgical fixation device 100 thereto, a suture (not shown) may also be used. Specifically, a suture may pass through tendon 20 at least once in predetermined locations 22 and may pass through and/or be anchored by surgical fixation device 100. Sutures passing through tendon 20 may also pass through and/or be secured through first suture loops 126 and second suture loops 128. In the example shown in FIG. 5, a suture may pass from first suture loop 126, through tendon 20, to second suture loop 128, and subsequently back through tendon 20 to a distinct first suture loop 126, and in similar patterns until the suture passes through tendon 20 and/or a desired number of suture loops 126, 128.

It should be noted that in FIG. 5, there is not a discrete separation between patellar tendon 20 and tibial tuberosity 18 as depicted. Here, the structures are such that tendon 20 blends into the bone at its insertion. It should be appreciated that there can be a separation that is more irregular than what is depicted. Further, the osteotomy depicted only includes the tibial tuberosity 18, but the osteotomy can be more posterior on the tibia, and there would be a clear space between the posterior aspect of the patellar tendon 20 and the tibial tuberosity 18. Thus, a larger portion of the proximal tibia anteriorly can be included in the osteotomy.

FIGS. 6-10 show additional non-limiting examples of surgical fixation device 100. It is understood that similarly numbered and/or named components may function in a substantially similar fashion. Redundant explanation of these components has been omitted for clarity.

In the non-limiting example shown in FIG. 6, bridge portion 122 may include a plurality of holes 124 formed therethrough. More specifically, the plurality of holes 124 may be formed through bridge portion 122, extending between first brace portion 102 and second brace portion 110 of surgical fixation device 100. Each hole 124, or a predetermined number of holes 124, formed through bridge portion 122 may receive a screw 130 (see, FIG. 4) to couple or affix bridge portion 122 to removed section 18 of tibia 10, as discussed herein.

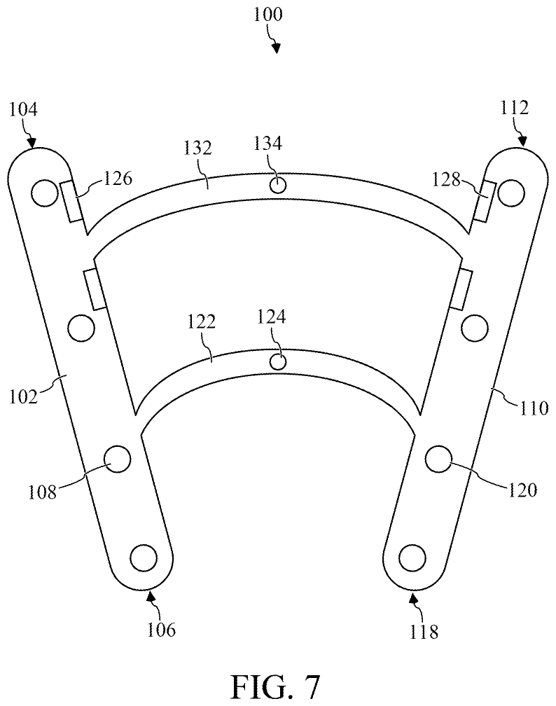

Turning to FIG. 7, another non-limiting example of surgical fixation device 100 is shown. In the non-limiting example, surgical fixation device 100 may also include a distinct bridge portion 132 extending between first brace portion 102 and second brace portion 110. Distinct bridge portion 132 may also be positioned adjacent to and separated from bridge portion 122. As shown, distinct bridge portion 132 may be positioned substantially adjacent and/or extend substantially between first end 104 of first brace portion 102 and first end 112 of second brace portion 110 of surgical fixation device 100. Distinct bridge portion 132 includes at least one hole 134 formed therethrough. In the example, a single hole 134 may be positioned or formed through distinct bride portion 132 centrally between first brace portion 102 and second brace portion 110. Although a single hole 134 is shown in distinct bridge portion 132, it is understood that distinct bridge portion 132 may include more holes 134 extending across the length of bridge portion 132.

FIGS. 8 and 9 shown a further non-limiting example of surgical fixation device 100. In the non-limiting example, first brace portion 102, second brace portion 110, and bridge portion 122 may be distinct portions or components. That is, first brace portion 102, second brace portion 110, and bridge portion 122 may be distinct such that bridge portion 122 is releasably coupled to each of first brace portion 102 and second brace portion 110, respectively. In the non-limiting example shown in FIG. 9, bridge portion 122 may include distinct holes 136 formed on opposing ends of bridge portion 122 that may align concentrically with one opening 108 of first brace portion 102 and one opening 120 of second brace portion 110. When affixing surgical fixation device 100 to tibia 10/removed section 18, screws 130 passing through one opening 108 of first brace portion 102 and one opening 120 of second brace portion 110, respectively, may also pass through holes 136 of bridge portion 122 to secure, couple, and/or connect bridge portion 122 to first brace portion 102 and second brace portion 110.

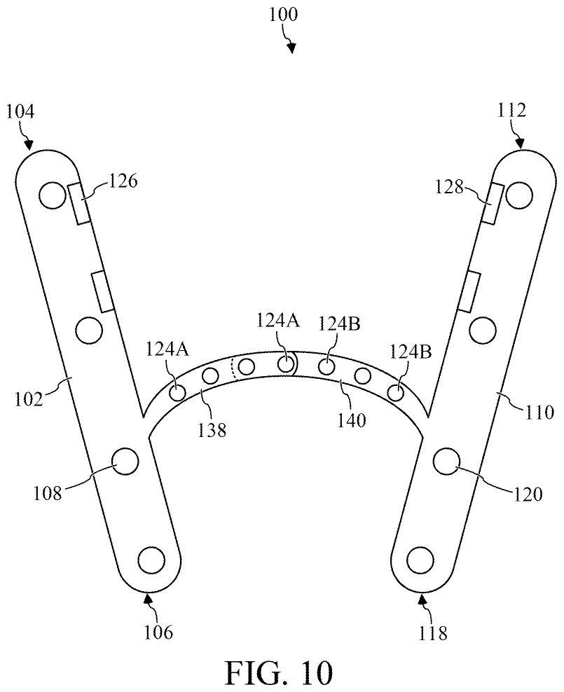

As shown in FIG. 10, surgical fixation device 100 may be formed from two distinct components. In the non-limiting example, bridge portion 122 may be formed as two distinct segments 138, 140. First segment 138 may be formed integral with first brace portion 102, where first segment 138 of bridge portion 122 may extend away from first brace portion 102. Additionally, second segment 140 may be firmed integral with second brace portion 110, and may extend away from second brace portion 110. As shown, second segment 140 may also extend toward first brace portion 102. In this example, first segment 138 of bridge portion 122 may be releasably coupled to second segment 140. More specifically, first segment 138 may include a first plurality of holes 124A formed therethrough, and second segment 140 may include a second plurality of holes 124B formed therethrough. The second plurality of holes 124B may be substantially (concentrically) aligned with the first plurality of holes 124A of first segment 138 to couple second segment 140 and first segment 138 of bridge portion 122. In a non-limiting example, screw(s) 130 may pass through concentrically aligned holes 124A, 124B of first segment 138 and second segment 140, respectively, to secure, couple, and/or connect the segments forming bridge portion 122, while also being embedded in removed section 18 and affixing surgical fixation device 100 to tibia 10 as discussed herein.

In the non-limiting examples shown and discussed herein with respect to FIGS. 6-10, it is understood that surgical fixation device 100 may be formed form any suitable material or any combination of materials having substantially malleable properties and/or characteristics. In the examples where surgical fixation device 100 is formed from at least two distinct components/portions, each portion may be formed from the same material, or alternatively may be formed distinct materials.

Thus, in operation, the device 100 is placed over the anterior aspect of the proximal tibia after the bone around the tibial tubercle/tuberosity has been cut for mobilization of the fragment and the attached patellar tendon 20 insertion. It is placed over the reduced fragment with the latter in its native (or desired) position in order to secure that fragment to the underlying native bone from which it was removed. The two plate portions 102, 104 of the device are placed on either side of the osteotomy fragment will be secured by unicortical screws (through the anterior cortex of the tibia) both medial and lateral to the fragment into the intact native bone. A transverse bridge portion 122 of the implant bridging between the two plates 102,104 will sit over the inferior aspect of the osteotomy fragment in order to secure the distal portion of that piece of the bone and prevent it from displacing anteriorly. With the device 100 in position, either wires or sutures 126,128 can then be passed through holes 124 on the anterior aspect of the plates at the same level from one portion of the plate to the other and then tied over the top of the tubercle osteotomy bone fragment in order to further secure the fragment in place. These holes may also be used (particularly at the most proximal, wider portion of the fragment) as a drill guide to allow the more proximal suture or wire to be passed through rather than around the bone fragment in order to prevent proximal displacement of the fragment. By holding the osteotomy fragment in this position, the bone-to-bone healing from fragment to native tibial bone is allowed to occur, allowing restoration of normal anatomy and extensor mechanism function of the knee (straightening out the knee).

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A surgical fixation device, the device comprising:
a first brace portion including:
a first end,
a second end formed opposite the first end, and
a first plurality of openings formed through the first brace portion, the first plurality of openings extending between the first end and the second end of the first brace portion;
a second brace portion positioned opposite the first brace portion, the second brace portion including:
a first end,
a second end formed opposite the first end, and
a second plurality of openings formed through the second brace portion, the second plurality of openings extending between the first end and the second end of the second brace portion; and
a bridge portion extending between the first brace portion and the second brace portion, the bridge portion including at least one hole formed therethrough.

2. The surgical fixation device of claim 1, wherein the bridge portion is formed integral with the first brace portion and the second brace portion, respectively.

3. The surgical fixation device of claim 1, wherein the at least one hole of the bridge portion includes a plurality of holes formed through the bridge portion and extending between the first brace portion and the second brace portion.

4. The surgical fixation device of claim 1, further comprising a distinct bridge portion extending between the first brace portion and the second brace portion, adjacent to and separated from the bridge portion.

5. The surgical fixation device of claim 4, wherein the distinct bridge portion includes at least one hole formed therethrough.

6. The surgical fixation device of claim 1, wherein the bridge portion is releasably coupled to each of the first brace portion and the second brace portion.

7. The surgical fixation device of claim 1, wherein the bridge portion includes:
a first segment formed integral with the first brace portion, the first segment extending away from the first brace portion; and
a second segment formed integral with the second brace portion, the second segment extending away from the second brace portion and toward the first segment.

8. The surgical fixation device of claim 7, wherein the first segment of the bridge portion is releasably coupled to the second segment.

9. The surgical fixation device of claim 7, wherein:
the first segment includes a first plurality of holes formed therethrough, and
the second segment includes a second plurality of holes formed therethrough, the second plurality of holes substantially aligned with the first plurality of holes of the first segment to couple the second segment and the first segment of the bridge portion.

10. The surgical fixation device of claim 1, wherein:
the first brace portion includes at least one first suture loop formed adjacent the first end, the at least one first suture loop formed adjacent the bridge portion, and
the second brace portion includes at least one second suture loop formed adjacent the first end, the at least one second suture loop formed adjacent the bridge portion.

11. The surgical fixation device of claim 10, wherein at least one of the first brace portion, the second brace portion, the bridge portion, the at least one first suture loop, or the at least one second suture loop is formed from a malleable material.

12. The surgical fixation device of claim 10, wherein:
the at least one first suture loop is formed integral with the first brace portion, and
the at least one second suture loop is formed integral with the second brace portion.

13. A method of securing an osteotomy fragment, comprising:
placing a surgical fixation device over an anterior aspect of a proximal tibia over an osteotomy fragment, the device comprising:
a first brace portion including:
a first end,
a second end formed opposite the first end, and
a first plurality of openings formed through the first brace portion, the first plurality of openings extending between the first end and the second end of the first brace portion;
a second brace portion positioned opposite the first brace portion, the second brace portion including:
a first end,
a second end formed opposite the first end, and
a second plurality of openings formed through the second brace portion, the second plurality of openings extending between the first end and the second end of the second brace portion; and
a bridge portion extending between the first brace portion and the second brace portion, the bridge portion including at least one hole formed therethrough;
wherein the placing of the device such that the first brace portion and second brace portion of the device are placed on either side of the osteotomy fragment;
securing the device to the tibia;
passing sutures through the first plurality of openings and second plurality of openings to thereby secure the osteotomy fragment to the tibia;
wherein the securing of the osteotomy fragment to the tibia promotes bone-to-bone healing of the osteotomy fragment to the tibia.

14. The method of claim 13, wherein the securing of the device to the tibia is screwing the device to the tibia.

15. A surgical fixation device, the device comprising:
a first means for bracing the device, including:
a first end,
a second end formed opposite the first end, and
a first plurality of fastening points formed through the first means for bracing, the first plurality of fastening points extending between the first end and the second end of the first bracing means;
a second means for bracing the device, the second means positioned opposite the first bracing means, the second brace means including:
a first end,
a second end formed opposite the first end, and
a second plurality of fastening points formed through the second bracing means, the second plurality of fastening points extending between the first end and the second end of the second bracing means; and
a bridging means extending between the first bracing means and the second bracing means, the bridging means including at least a bridge fastening point.

16. The device of claim 15, wherein the bridging means is formed integrally with the first bracing means and the second bracing means.

17. The device of claim 15, wherein the bridging means further includes a plurality of bridge fastening points.

18. The device of claim 15, furthering including a first fastener attached to the first plurality of fastening points and second plurality of fastening points.

19. The device of claim 15, further including a bone fastening means for selectively fastening the device to a bone.

20. The device of claim 18, further including a second fastener attached to the bridge fastening point.
